(19) United States
(12) Patent Application Publication
(10) Pub. No.: US 2006/0116693 A1
(43) Pub. Date: Jun. 1, 2006

(54) Apparatus and method for stone capture and removal

(76) Inventors: William Bruce Weisenburgh II, Maineville, OH (US); Robert P. Gill, Mason, OH (US); James W. Voegele, Cincinnati, OH (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/290,999

(22) Filed: Nov. 30, 2005

Related U.S. Application Data
(60) Provisional application No. 60/632,089, filed on Dec. 1, 2004.

(51) Int. Cl. A61B 17/22

(52) U.S. Cl. 606/128

(57) ABSTRACT

A system and method to be used in ultrasonic or other types of lithotripsy of a stone in a ureter, the system including a catheter having a stone probe tip capable of transmitting stone reducing energy. The catheter can include an expandable funnel section adjacent to the probe tip, such that expansion of the expandable funnel section can dislodge a stone by pushing back on the ureter wall expanding it slightly. The funnel section also being capable of pooling some urine in the ureter to be used as an ultrasonic transmission media. The stone probe can be connected to a source of energy capable of driving the probe tip to deliver energy to break apart the stone.

found in the lower ureter or stones impacted in the upper ureter for example. IL techniques, however, typically require use of general anesthetic, guidewires for getting a basket past the stone, stent placement, imaging equipment and the ability to respond to ureter perforations that may occur. For these reasons IL requires the use of a surgical suite to perform the procedure. One prior art approach to IL is transurethral lithotripsy. Transurethral lithotripsy involves using a fiber optic ureterscope to place an ultrasonic probe adjacent to a stone. The ureterscope is used to guide the placement of the probe through the bladder and up the ureter. Once placed against the stone an ultrasonic generator can drive the probe to produce U/S energy to destroy the stone. Another alternative is to use a laser to disintegrate the stone.

A problem with prior art approaches to intracorporeal treatment of ureter stones is that the stone is not captured during treatment. The stone can be pushed up the ureter toward the kidney in response to the efforts to treat it. The stone can also move to the side of the catheter and wedge between the ureter wall and the end of the catheter. In some cases the stone may be difficult to image and a catheter containing fiber optic equipment might move the stone. In other cases a catheter containing the contact ultrasonic (U/S) probe might move the stone. Movement of the stone makes treatment difficult and increases the risk of injury to the interior of the ureter. This is one problem that leads to intracorporeal lithotripsy being a more expensive in-patient treatment requiring a surgical suite as opposed to an out patient treatment.

Another problem caused by stone movement is that most treatment techniques are most effective when the stone location is precisely known and can be held. If the stone is moving in response to the treatment and in response to body changes then the lithotripsy treatment will be more difficult and time consuming to perform. Another factor worthy of consideration is the stone and prior art treatment device size. A too large stone and/or device requires a stent to be placed in the ureter to overcome strictures preventing urine flow.

It can be seen that there is a need for an improved apparatus and method to treat stones in the ureter. In can be seen that there is a need for a treatment system and method where a stone will not move during treatment. It can also be seen there is a need for a stone treatment procedure that can be performed without getting behind the stone.

### BRIEF SUMMARY OF THE INVENTION

The present invention relates to a system and method to be used in stone management within a ureter. The system can include a catheter having a probe tip capable of transmitting ultrasonic energy, an expandable device adjacent to the probe tip and a source of energy capable of driving the probe tip to deliver ultrasonic energy to break apart the stone.

In another aspect of the invention, an inflatable balloon can cause pooling of urine fluid in the ureter such that the pooled urine fluid can act as a medium to transmit ultrasonic energy from the probe to the stone.

In yet another aspect, the system includes a catheter having a probe tip capable of transmitting disintegration energy to a stone. The system can include an expandable portion adjacent to said probe tip, the expandable portion including fingers that can push outward against a wall of the ureter to release a stone lodged in the ureter. The system can also include a source of energy capable of driving the probe tip to break apart the stone.

In a further aspect of the invention, a method of performing ultrasonic lithitripsy is disclosed including the steps of placing a catheter having an ultrasonic probe in the ureter adjacent to a stone. The method can include a step of expanding a device to pool urine fluid and to cause movement of the stone. The step can include expanding the device adjacent to the stone to hold back the wall of the ureter and allow a stone to dislodge from the ureter. Under pressure from the flow of urine, peristaltic pressure from the urine or gravity, the stone will move into the expanded funnel where it can be held in a known position for treatment and allows the use of an ultrasonic energy source to drive the probe to break apart the stone.

In yet a further aspect of the invention, the device and method may be used to remove smaller stones by grasping them and pulling them out of the ureter without breaking them.

The present invention is useful in open or endoscopic surgeries as well as robotic-assisted surgeries.

Further features and advantages of the present invention will become readily apparent from the following detailed description, the accompanying drawings, and the appended claims.

### BRIEF DESCRIPTION OF THE FIGURES

FIG. 1 is a view of the components of the stone treatment catheter distal end;

FIG. 2 is a cross-sectional view of the distal end of the device presented to a stone;

FIG. 3 is an enlarged view of the distal end in accordance with the invention;

FIGS. 4a-e illustrate the sequential method steps of use for one aspect of the invention;

FIG. 5 is a partial sectional view of an alternate embodiment of the invention;

FIG. 6 is a partial sectional view of the alternate embodiment shown in FIG. 5 and with an expanded distal end; and

FIG. 7 illustrates a cut-away view of one aspect of the invention.

### DETAILED DESCRIPTION OF THE INVENTION

Before explaining the present invention in detail, it should be noted that the invention is not limited in its application or use to the details of construction and arrangement of parts illustrated in the accompanying drawings and description. The illustrative embodiments of the invention may be implemented or incorporated in other embodiments, variations and modifications, and may be practiced or carried out in various ways. Furthermore, unless otherwise indicated, the terms and expressions employed herein have been chosen for the purpose of describing the illustrative embodiments of the present invention for the convenience of the reader and are not for the purpose of limiting the invention.

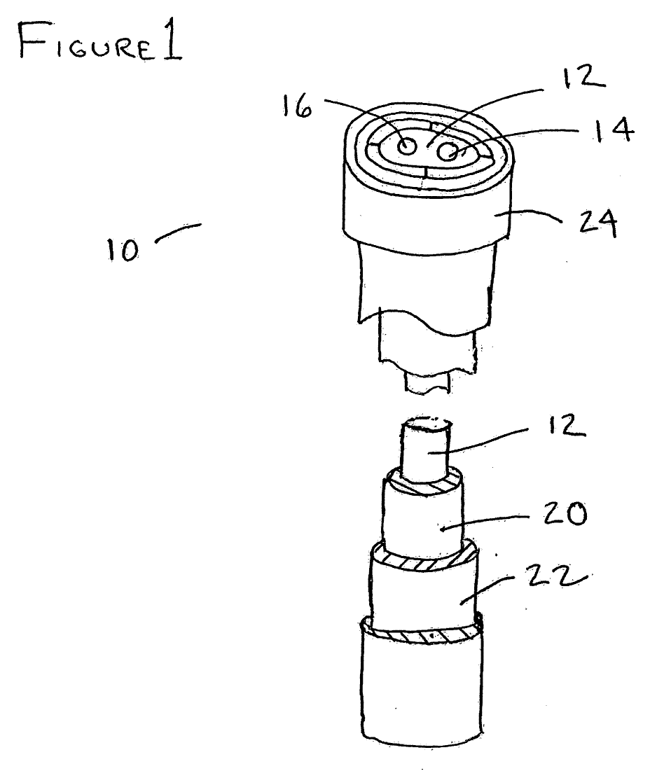

The novel features of the invention are set forth with particularity in the appended claims. The invention itself, however, both as to organization and methods of operation, together with further objects and advantages thereof, may best be understood by reference to the following description, taken in conjunction with the accompanying drawings in which FIG. 1 shows a partial cross section of the catheter system 10. The catheter system 10 includes a center section 12, which can include a fiber optic 14 to view the treatment area and a second fiber 16 to carry light to an operation site.

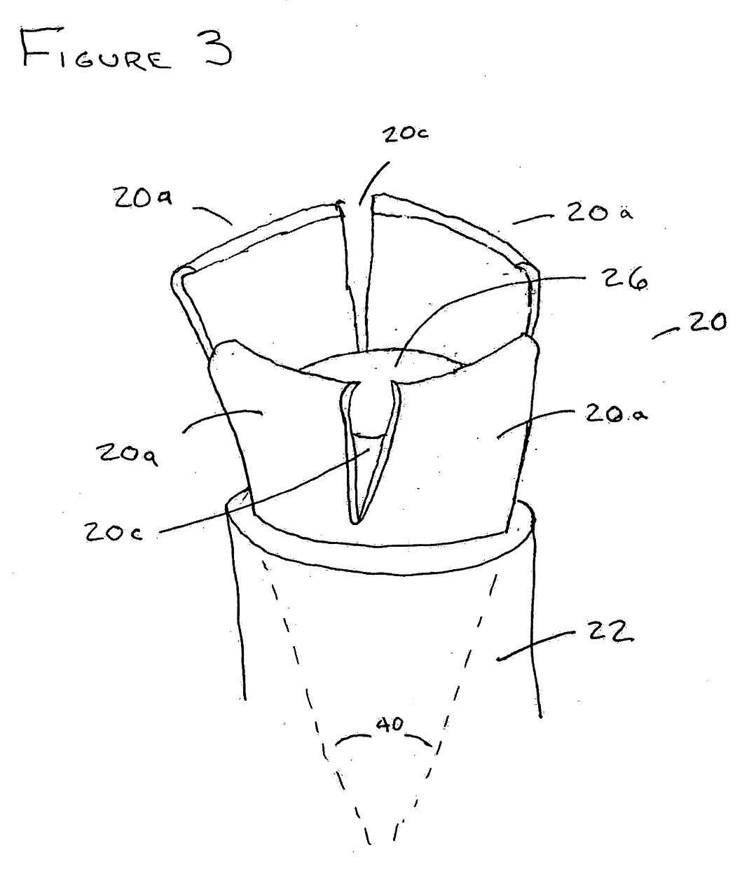

The catheter system 10 further comprises a funnel section 20, a catheter sheath section 22 and a balloon section 24. Each section 20-24 can be in the shape of a hollow cylinder. The fiber optic section 12 can be slid out of the funnel section 20 such that a stone treatment probe (26 in FIG. 3) of about the same diameter can be inserted into the funnel section 20.

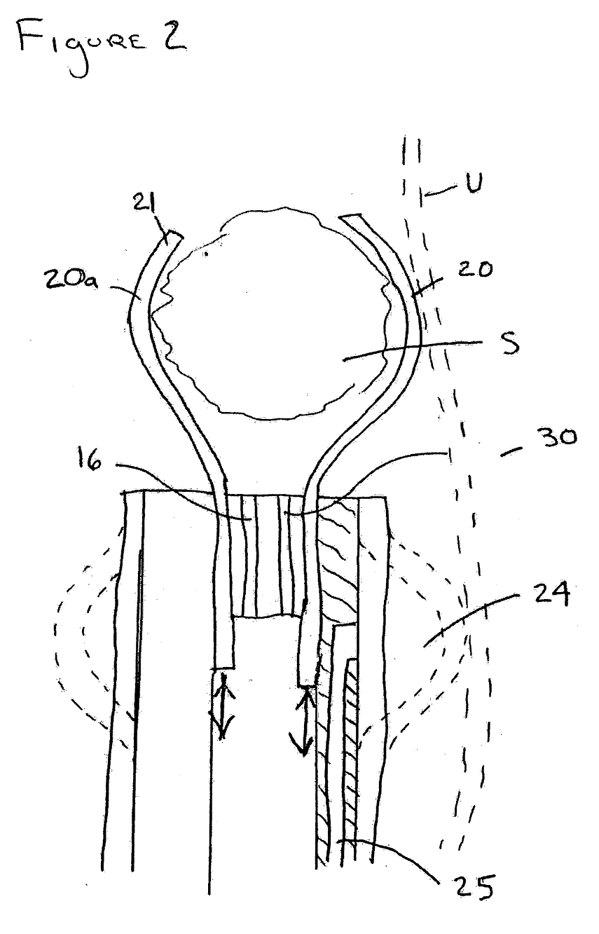

FIGS. 2 and 3 show the catheter system 10 and its distal tip 30 in operation. Inside the ureter U the balloon section 24 is expanded by air or other biocompatible fluids through channel 25. The expansion of the balloon section 24 slightly expands the ureter U wall and dams the urine causing the stone S, which had been held by the ureter U, to loosen. Once the balloon section 24 expands, the funnel section 20 can be moved forward, the fingers 20a are exposed and radially expand towards a free state, shown in FIG. 3, further expanding the ureter wall to release the stone S.

The fingers 20a are formed to normally, in a free state, have a funnel shape 40 (shown in FIG. 3) the larger end of which can be larger than the inside diameter of the sheath 22. The tips of fingers 20a, have curves 21 that curve back into the center axis of the catheter system 10 to grip as can be seen in FIGS. 2 and 3. When the fingers 20a are in the sheath section 22, the fingers 20a are collapsed to form a cylinder funnel section 20 as shown in FIG. 1. When the funnel section 20 is slid forward to expose the fingers 20a, the fingers can flare out to the normal funnel shape 40 forming a gap 20c between fingers.

FIG. 3 shows more details including all the fingers of the funnel section 20. Four fingers 20a can be separated by four gaps 20c. With the funnel section 20 pushed forward, the fingers 20a can assume their normal free state positions forming a funnel shape 40. Flared out in the ureter U as shown in FIG. 2, the funnel shape 40 can hold the stone S in a steady, known position. FIG. 3 shows that the center section 12, containing fiber optics 14 and 16, has been removed and has been replaced with an catheter stone treatment probe 26 that can be driven by a power supply (not shown) to reduce the size of stone S. The center section 12 is removed from the funnel section 20 by sliding it out and the catheter stone treatment probe 26 is slid into the funnel section 20 replacing the center section 12. Pulling the center 12 section out can create a suction that will further pull the stone S into the fingers 20a if it is not fully held by the actions of the balloon 24 and finger section 20.

In operation, the catheter 10 is guided through a bladder and into the ureter U using fiber optics 14 and 16. The tip 30 is positioned adjacent the stone S. The balloon 24 is expanded and the funnel section 20 is pushed forward exposing fingers 20a. The expansion of the balloon 24 loosens the stone S, the release of the fingers 20a and pressure from urine dammed by the balloon 24 can release the stone S from the ureter and the stone S moves into the funnel shape 40. With the balloon 24 expanded, the dammed urine adjacent the tip 30 can act as a medium to transmit ultrasonic energy to the stone S.

With the stone S in place in the funnel shape 40, the center section 12 containing fiber optics 14 and 16 can be slid out of the funnel section 20 and a catheter section (not shown) tipped by stone probe 26 can be inserted into the funnel section 20. Ultrasonic energy or other means can be applied through the stone probe 26 to disintegrate the stone S. A representative ultrasonic transducer for use is disclosed in U.S. Pat. Nos. 6,049,159; 6,050,943; and 6,120,452; each of which is incorporated herein by reference. Alternatively, a small stone, gripped by fingers 20a may be moved to any desired location within the ureter or pulled out of the ureter without use of ultrasonic or other lithotripsy techniques.

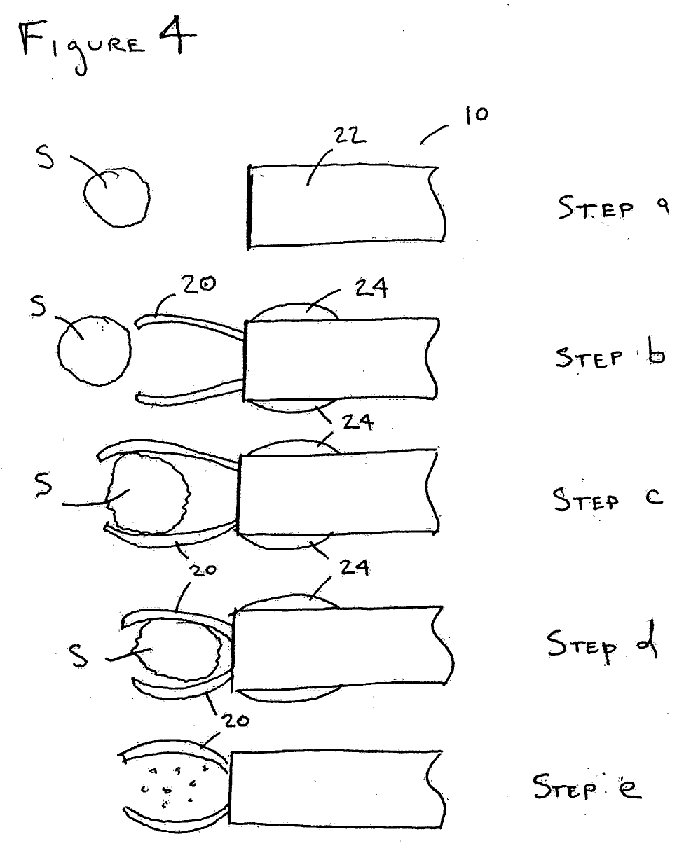

FIGS. 4a-e show a simplified view of the operation of the device 10. The device 10 is presented to a stone S in FIG. 4a. In FIG. 4b, the balloon 24 is inflated and the finger section 20 is extended toward the stone S. The stone S is released from the wall of the ureter U (not shown here) and moves toward the device 10. In FIG. 4c the stone S is grasped within the fingers 20. In FIG. 4d the stone S can be pulled closer to the device by retracting the finger section 20. The center section is removed and replaced by the stone treatment probe. In FIG. 4e the stone S is broken apart. As mentioned for a small stone S, FIG. 4d could be followed by an alternate step of simply pulling the stone S out. The stone S could be addressed by ultrasonic energy or by laser for example. As stone treatment proceeds, it is possible to re-introduce the center section 12 to view progress between applications of ultrasonic energy.

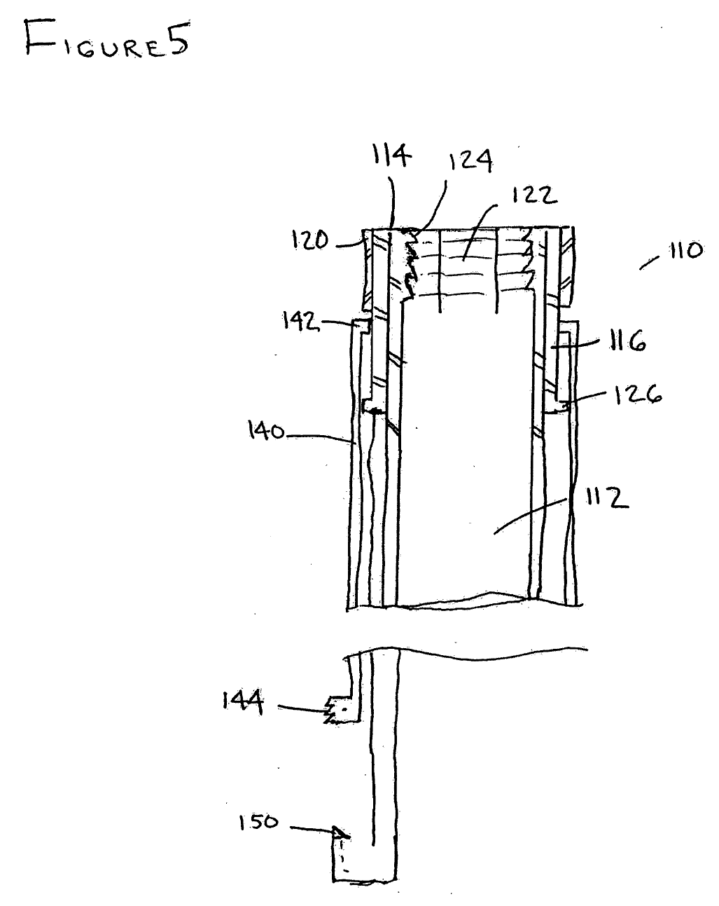

FIG. 5 shows a cross sectional view of an alternate embodiment of the device. A catheter 110 can include a catheter inner funnel section 112 connected by hinge portion 114 to outer funnel section 116. The arrangement includes an outer elastic band 120 surrounding fingers 122. In this embodiment, the fingers 122 can include gripping teeth 124 that can improve the ability of the fingers 122 to grip a stone S. The outer funnel section 116 can include a stop ring 126. A sheath portion 140 can include a cooperating stop ring 142. The sheath portion 140 can include a ratcheted stop ring 144 and the inner funnel section 112 can include an annular detent 150.

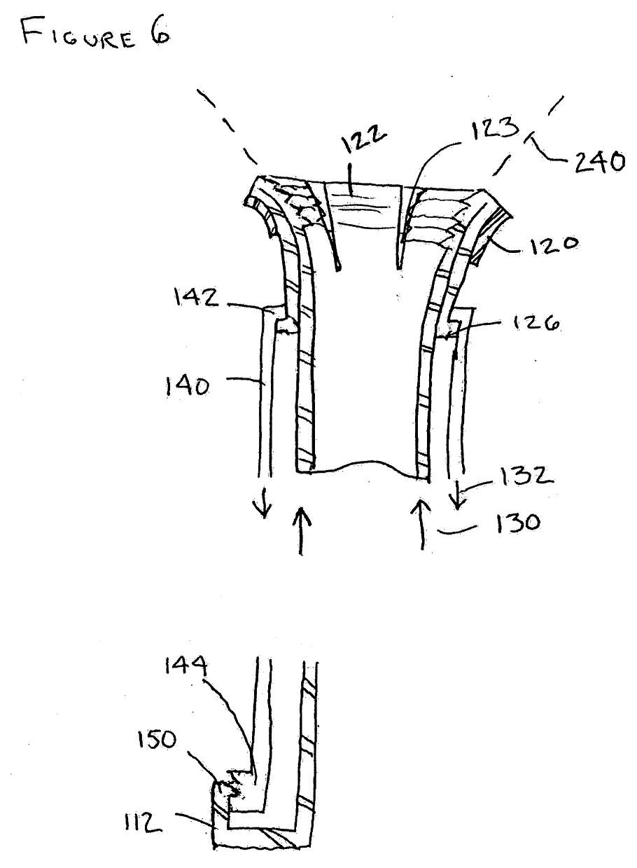

FIG. 6 shows a cross sectional view of the embodiment of FIG. 5 in an expanded position. Sheath portion 140 has been pulled down so that the stop rings 126 and 142 meet. As the operator pulls further, arrows 130 and 132 show lines of force that will be applied. An upward force 130 is placed on the inner funnel section 112 while a downward force 132 of equal magnitude is placed on the outer funnel section 116. The result of the forces 130,132 is that the fingers 122 open up to form a funnel shape 240 indicated by dashed lines. The fingers 122 are separated by slits 123 that allow the relative forces 130/132 to deform the fingers 122. The elastic band 120 is provided to prevent injury to the inner wall of the ureter U that might be pinched when the slits 123 closed if the band 120 were not present. Once the relative forces 130,132 are removed the fingers 122 can be returned to their original positions as shown in FIG. 5. During a procedure to reduce a stone S the forces 130,132 can be maintained by a clamp mechanism created by ratcheted stop ring 144 and detent 150. As can be seen, the teeth of ratcheted stop ring 144 will maintain the inner 112 and outer 116 funnel sections in their biased relationship until an operator releases it. Ultrasound can transmit through inner funnel 112 and fingers 122. Fingers 122 can serve to transmit sound energy to the stone. The ratcheted stop ring 144 can be progressively stepped further into the detent 150 to further open the fingers 122 and widen the funnel 240.

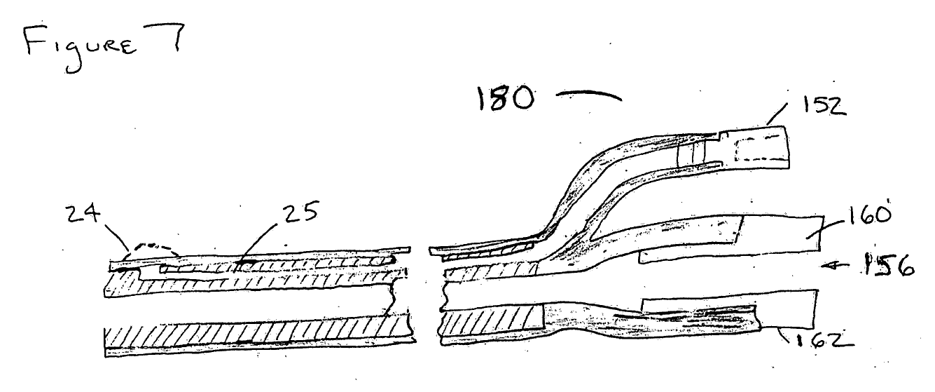

FIG. 7 illustrates the proximal end 180 opposite the distal tip 30 of the catheter 10. The passage 25 provides air from a fitting 152 to the balloon 24. A working space 156 allows for the placement and manipulation of sections such as the center section 12. Fittings 160 and 162 allow for the locking of any section in place and for the establishment of location datum.

In operation the embodiment of FIGS. 5-7 is inserted through the bladder using a cystoscope (not shown) into the ureter U. Once in the ureter, the catheter 110 can be positioned near or adjacent to a stone as appropriate. Though not shown, fiber optic devices can be held in the inner diameter of the inner funnel section 112 and the fiber optics can be used to guide the catheter 110 through the ureter to a stone. Once presented to a stone, the inner funnel section 112 can be biased relative to the outer funnel section 116. The relative forces, as demonstrated by arrows 130 and 132 in FIG. 5, will cause the flexible fingers 122 to flare outward where the fingers 122 can contact the wall of the ureter U and dislodge a stone as shown for the first embodiment in FIG. 2. The flared fingers 122 can form a funnel shape 240 that can capture and hold a stone using gripping teeth 124. The ratcheted stop ring 144 can hold the fingers 122 in a desired position so that the operation can proceed.

Similar to the first embodiment, once the catheter 110 is presented to a stone and the fingers 122 flared out, the stone can move in response to pressure from the flow of urine, peristaltic pressure or gravity to become lodged and held in the funnel shape 240. Once the stone is positioned in the funnel 240, the fiber optic portion of the catheter can be slid out and a stone treatment probe 26 (FIG. 3) can be slid into the workspace 156 and inner funnel section 112. The stone treatment probe 26 can then be used to break apart the stone. The band 120 can protect the wall of the ureter U during the procedure. Like the first embodiment, the expanded funnel 240 can be used to control the rate of flow of urine through the ureter so that a pool of urine fluid can be formed to act as a medium to transmit ultrasonic energy.

Though the embodiment of FIGS. 5 and 6 do not require a balloon as shown in FIG. 7 it will be understood by those skilled in the art that a balloon could be used in cooperation with this embodiment to pool urine for example. Though not shown, it will be understood that irrigation fluid such as saline solution could be used to irrigate the stone as it is disintegrated and that irrigation or urine fluid could be drained away from the stone to carry away process byproducts. It will also be understood that the ureter stone treatment probe 26 could be of any conventional stone management type such as ultrasonic, laser, impact, or spark gap. Any method of intra-ureter stone treatment could be used. It will also be understood that while 4 fingers are shown forming the funnel shape in the disclosed embodiments, any number of fingers could be used. Further though shown as formed by fingers the funnel shape could be formed from a variety of shape memory or deformable elements that could be released to form a funnel shape. It will also be understood that the funnel could assume a variety of shapes similar to a cone and that a variety of shapes and materials could be used on the fingers to help them grip the stone.

While the present invention has been illustrated by description of several embodiments, it is not the intention of the applicant to restrict or limit the spirit and scope of the appended claims to such detail. Numerous variations, changes, and substitutions will occur to those skilled in the art without departing from the scope of the invention. Moreover, the structure of each element associated with the present invention can be alternatively described as a means for providing the function performed by the element. Accordingly, it is intended that the invention be limited only by the spirit and scope of the appended claims.

What is claimed is:

1. A system to be used in lithotripsy of a stone in a ureter, said system comprising;
a catheter having a probe tip capable of transmitting energy;
an expandable portion adjacent to said probe tip;
said expandable portion including fingers that can push outward against a wall of the ureter; and
a source of energy capable of driving said probe tip to deliver energy to disintegrate said stone.

2. A method of performing ultrasonic lithitripsy on a stone in ureter including the steps of;
placing a catheter having an ultrasonic probe in the ureter adjacent to a stone;
capturing said stone by expanding a portion of said catheter adjacent said probe to expand against a wall of said ureter;
at least partially blocking a flow of fluid in said ureter to act as a medium for transmitting ultrasonic energy; and
using a source of ultrasonic energy to drive the probe to break apart the stone.

3. A catheter to be used in lithotripsy of a stone in a ureter, said catheter including;
a probe tip capable of transmitting ultrasonic energy;
an expandable portion adjacent to said probe tip;
said expandable portion including a funnel that can push outward against a wall of the ureter; and
a source of energy capable of driving said probe tip to deliver ultrasonic energy to break apart said stone.
